(19) United States
(12) Patent Application Publication
(10) Pub. No.: US 2024/0164811 A1
(43) Pub. Date: May 23, 2024

(54) NASAL AND EAR FOREIGN BODY EXTRACTOR

(71) Applicant: Eon Meditech Pvt. Ltd., Utran, Surat, Gujarat (IN)

(72) Inventor: Brian Kamradt, Martinsville, IN (US)

(73) Assignee: Eon Meditech Pvt. Ltd., Utran, Surat, Gujarat (IN)

(21) Appl. No.: 18/283,561

(22) PCT Filed: May 20, 2022
(86) PCT No.: PCT/US2022/030219

§ 371 Date: Sep. 22, 2023

Related U.S. Application Data
(60) Provisional application No. 63/164,947, filed on Mar. 23, 2021.

(51) Int. Cl. A61B 17/50; A61B 17/24; A61F 11/00; A61M 25/10

(52) U.S. Cl. CPC A61B 17/50; A61B 17/24; A61F 11/006; A61B 2017/246; A61M 25/10

(57) ABSTRACT

Nasal and ear foreign body extracting device. The present disclosure includes disclosure of a device, comprising a bulb comprising a walled chamber portion having a nipple coupled thereto or formed as part thereof, a catheter configured to engage the nipple of the bulb at a first catheter end, and a balloon positioned at a second catheter end opposite the first catheter end, wherein when the bulb is compressed, air from within the bulb is pushed from the bulb, through the catheter, and into the balloon to inflate the balloon.

### PRIORITY

The present application is related to, and claims the priority benefit of, U.S. Provisional Patent Application Ser. No. 63/164,947, filed Mar. 23, 2021, the contents of which are incorporated herein directly and by reference in their entirety.

### TECHNICAL FIELD

The present disclosure relates to an extracting device for removing objects from the nasal and ear passages.

### BACKGROUND

The present disclosure is directed toward an extracting device, and more particularly, to an extracting device for removing foreign objects from the nasal and ear passages. Human nature leads children, and sometimes adults, to put objects where they don't belong, such as within nasal cavities and ear canals, causing discomfort and injury. Forceps are the most common device for removing such foreign objects from the blocked passageway. However, forceps can be damaging to the sensitive tissues which often make up these passageways and thus, further damage may result during the attempted removal of the object. This method is often lengthy and traumatic for a child, and it may be unsuccessful, resulting in surgery.

Medical technology does include devices for removing objects from human body passages. These devices generally include a catheter having an eccentric balloon attached to its distal end. In operation, the catheter is inserted into the nasal cavity or ear canal so that the balloon in a deflated state moves behind an object to be removed. The balloon is then inflated and moved into contact with the object to be removed.

Embodiments of such an extractor are disclosed in U.S. Pat. No. 5,454,817. One major drawback of this extractor is of a mechanically complicated and costly construction. In the extractor embodiments therein, the distal and proximal balloons are connected with a catheter and make up a closed circuit allowing air to be transferred from one balloon to the other when one or the other is compressed and relies on a sealed leak proof construction. The drawback of this design is the maintaining of this sealed and leak proof construction.

### BRIEF SUMMARY

It is an object of the present disclosure to provide an effective nasal and ear foreign body extracting device which is easily manipulated and held with one hand. Another object of this disclosure is to provide a foreign object extracting device which is simple and safe to use for home care as well as physician care. Yet another object of the present disclosure is to provide a foreign object extracting device which is inexpensive to manufacture and purchase.

The present disclosure includes disclosure of a device, comprising a bulb comprising a walled chamber portion having a nipple coupled thereto or formed as part thereof, a catheter configured to engage the nipple of the bulb at a first catheter end, and a balloon positioned at a second catheter end opposite the first catheter end, wherein when the bulb is compressed, air from within the bulb is pushed from the bulb, through the catheter, and into the balloon to inflate the balloon.

The present disclosure includes disclosure of a device, wherein the bulb further comprises a valve.

The present disclosure includes disclosure of a device, wherein when the bulb is compressed, the valve closes so that air from within the bulb is pushed into the catheter to inflate the balloon instead of escaping from the valve.

The present disclosure includes disclosure of a device, wherein when the bulb is not compressed, the valve is open.

The present disclosure includes disclosure of a device, wherein the bulb comprises a flexible, semi-rigid material so to permit the bulb to be compressed and released, returning to a native configuration when released.

The present disclosure includes disclosure of a device, wherein the balloon is sized and shaped to fit within a nasal cavity or an ear cavity when fully inflated.

The present disclosure includes disclosure of a method, comprising inserting part of a device of the present disclosure into a nasal cavity or an ear cavity so that the balloon is positioned distal to an object present within said cavity, inflating the balloon by compressing the bulb, and retracting the device from the nasal cavity or the ear cavity while maintaining compression of the bulb so that the balloon pulls the object out of the nasal cavity or the ear cavity.

The present disclosure includes disclosure of a method, comprising releasing compression of the bulb to deflate the balloon.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows an isometric view of a nasal and ear foreign body extracting device, according to at least one embodiment of the present disclosure;

FIG. 2 shows an isometric view of a nasal and ear foreign body extracting device, according to at least one embodiment of the present disclosure;

FIG. 3 shows a side view of a nasal and ear foreign body extracting device, according to at least one embodiment of the present disclosure; and

FIG. 4 shows a section view of the nasal and ear foreign body extracting device along axis A-A shown in FIG. 3, according to at least one embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

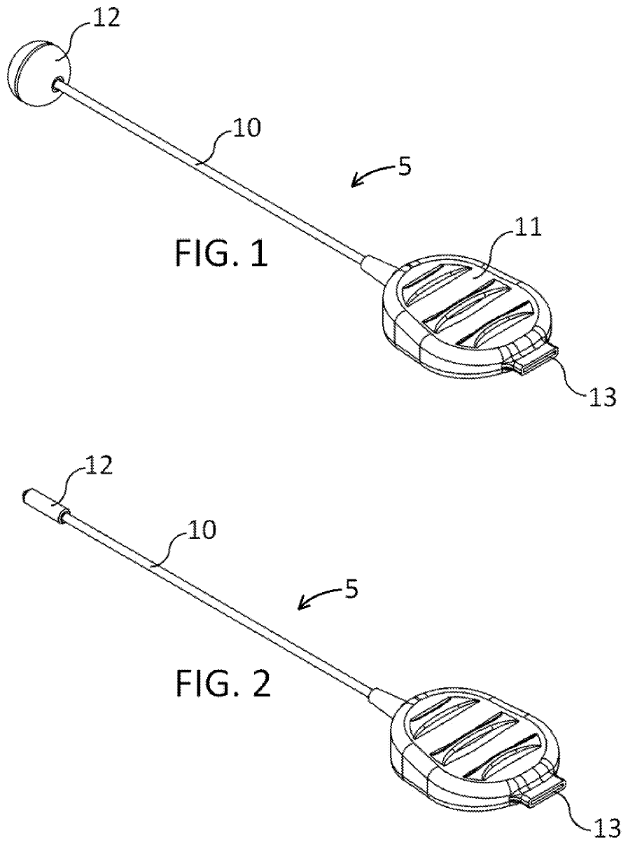

Referring now to the drawings in detail, shown in FIG. 1, an exemplary extracting device 5 of the present disclosure is shown in its inflated state, which includes a bulb 11 connected on one end to an elongated tubular rod or catheter 10, and a balloon 12 connected to the other end (an opposite second end) of the rod or catheter 10. Balloon 12 is shown in FIG. 1 in its inflated state.

An exemplary extracting device 5 of the present disclosure is shown in FIG. 2 in its deflated state, which also includes a bulb 11 connected on one end to a flexible elongated tubular rod or catheter 10, and a balloon 12 connected to the other end (an opposite second end) of the rod or catheter 10. Balloon 12 is shown in FIG. 2 in its deflated state.

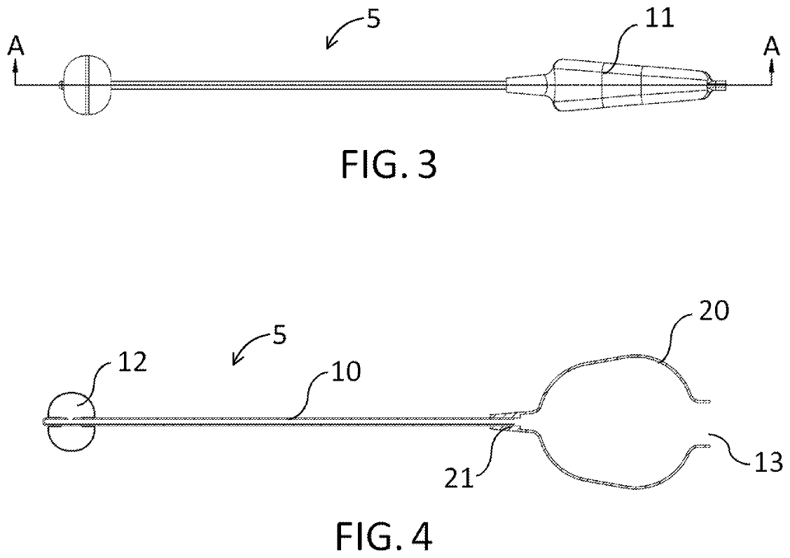

Bulbs 11 of the present disclosure are used for inflating and deflating balloon 12 with air. Bulbs 11 therefore are constructed from semi-rigid flexible rubber, compliant plastic, or a similar resilient material. Bulbs 11 comprise a walled chamber portion 20 defining/forming an internal chamber therein, and a nipple 21 for connection to catheter 10. Bulbs 11 also include a valve opening 13 for letting air in or out, such as shown in FIG. 3. Bulbs 11 may also be formed by other configurations or using other objects having a hollow center. Bulbs 11 may be compressed by squeezing, but because of the semi-rigid yet flexible nature of walled chamber portion 20, bulbs 11 will return to their original shape after compression.

Bulbs 11 are in fluid communication with catheters 10 in order to move air therethrough. Balloons 12 are also in fluid communication with catheters 10 of devices 5 of the present disclosure. Balloons 12 are positioned and connected with catheters 10 so as to receive the air from bulbs 11 and, as a result, become inflated.

The volume of bulb 11 is such that upon one compression of bulb 11, balloon 12 will inflate to or below a maximum volume of inflation of the balloon. In order to maintain the inflation of balloon 12, bulb 11 must be held in a compressed state The need to hold compression acts as a safety measure for avoiding over-inflation of balloon 12, as if the compression is released, balloon 12 will deflate, thus avoiding inflation of balloon 12 to a volume exceeding the volume of air in bulb 11 able to be transferred to balloon 12 through catheter 10.

An exemplary bulb 11 of the present disclosure is attached to the catheter 14 via a nipple 21, or, for example, formed as a unitary item along with catheter 10. In embodiments with a nipple 21, said nipple 21 can be formed along with walled chamber portion 20 during the molding process. Nipple 21 extends outwardly from walled chamber portion 20 for connection to catheter 10. Nipple 21 is also hollow, and defines an output port of bulb 11 for release of the air from walled chamber portion 20 of bulb 11 to the catheter 10.

Bulbs 11 of the present disclosure have a valve 13 for air inlet and outlet and for pumping air through said catheter 10, allowing for the inflation and deflation of said balloon 12 as desired. Valves 13 are configured to allow air into bulb 11 when bulb 11 is in a native, inflated state, and effectively close when bulb 11 is compressed, forcing air from within bulb 11 through catheter 10 and into balloon 12 to inflate balloon 12. Upon release of compression of bulb 11, air from balloon 12 flows back through catheter 10 and into bulb 11 and/or through valve 13 into the immediate atmosphere of bulb 11.

When in use, a balloon 12 end of device 5 can be inserted into the nasal or ear cavity so that it is present distal to, or relatively “past,” and object present therein. After balloon 12 is present distal to said object (so that bulb 11 is proximal to, or relatively “before,” said object), balloon 12 can be inflated by compressing bulb 11, which forces air from within bulb 11 out of bulb 11, through catheter 10, and into balloon 12 to inflate the same. Device 5 can then be withdrawn from the nasal or ear cavity while bulb 11 remains compressed (so that balloon 12 remains inflated), effectively pulling the object in said cavity with device 5 to remove said object. Once said object is removed, bulb 11 can be released to deflate balloon 12. Balloon 12 is configured so that when it is inflated within the nasal or ear cavity, the inflated balloon 12 conforms to surfaces of said cavity.

While various embodiments of devices and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device, comprising:
a bulb comprising a walled chamber portion having a nipple coupled thereto or formed as part thereof;
a catheter configured to engage the nipple of the bulb at a first catheter end; and
a balloon positioned at a second catheter end opposite the first catheter end;
wherein when the bulb is compressed, air from within the bulb is pushed from the bulb, through the catheter, and into the balloon to inflate the balloon.

2. The device of claim 1, wherein the bulb further comprises a valve.

3. The device of claim 2, wherein when the bulb is compressed, the valve closes so that air from within the bulb is pushed into the catheter to inflate the balloon instead of escaping from the valve.

4. The device of claim 2, wherein when the bulb is not compressed, the valve is open.

5. The device of claim 1, wherein the bulb comprises a flexible, semi-rigid material so to permit the bulb to be compressed and released, returning to a native configuration when released.

6. The device of claim 1, wherein the balloon is sized and shaped to fit within a nasal cavity or an ear cavity when fully inflated.

7. A method, comprising:
inserting part of the device of claim 1 into a nasal cavity or an ear cavity so that the balloon is positioned distal to an object present therein;
inflating the balloon by compressing the bulb; and
retracting the device from the nasal cavity or the ear cavity while maintaining compression of the bulb so that the balloon pulls the object out of the nasal cavity or the ear cavity.

8. The method of claim 7, further comprising:
releasing compression of the bulb to deflate the balloon.
